(19) United States
(12) Patent Application Publication
(10) Pub. No.: US 2005/0149142 A1
(43) Pub. Date: Jul. 7, 2005

(54) Gastric stimulation responsive to sensing feedback

(76) Inventor: Warren L. Starkebaum, Plymouth, MN (US)

Correspondence Address: MEDTRONIC, INC., 710 MEDTRONIC PARKWAY NE, MS-LC340, MINNEAPOLIS, MN, 55432-5604, US

(21) Appl. No.: 10/768,995

(22) Filed: Jan. 30, 2004

Related U.S. Application Data
(60) Provisional application No. 60/535,144, filed on Jan. 7, 2004.

(51) Int. Cl. A61N 1/18

(52) U.S. Cl. 607/40

(57) ABSTRACT

In general, the invention is directed to methods and devices for monitoring one or more physiological parameters that reflect the activity of the stomach of a patient, and generating an electrical stimulation signal to induce symptoms of gastroparesis. The induced symptoms of gastroparesis may reduce a patients desire to consume large portions of food and thus provide an effective treatment for obesity.

### RELATED PATENTS

This application claims the benefit of U.S. Provisional Application to Starkebaum, entitled, “GASTRIC STIMULATION RESPONSIVE TO SENSING FEEDBACK,” Ser. No. 60/535,144, filed Jan. 7, 2004 (Attorney Docket No. P-9905.00).

### FIELD OF THE INVENTION

The invention relates to medical devices and methods and, more particularly, to medical devices and methods for electrical stimulation of the stomach.

### BACKGROUND

Obesity is a major health concern in the United States as well as other western countries. A significant portion of the population is overweight with the number increasing every year. Obesity is one of the leading causes of preventable death. Obesity is associated with several co-morbidities that affect almost every body system. Some of these co-morbidities include: hypertension, heart disease, stroke, high cholesterol, diabetes, coronary disease, breathing disorders, sleep apnea, cancer, gallstones, and musculoskeletal problems. An obese patient is also at increased risk of developing Type II diabetes.

Multiple factors contribute to obesity, including physical inactivity and overeating. Existing therapies include diet, exercise, appetite suppressive drugs, metabolism enhancing drugs, surgical restriction of the gastric tract, and surgical modification of the gastric tract. These therapies may result in little or no weight loss up to weight loss of nearly 50% of initial body weight.

Gastroparesis is an adverse medical condition in which normal gastric motor function is impaired. Gastroparesis is also called delayed gastric emptying as the stomach takes too long to empty its contents. Typically, gastroparesis results from muscles of the stomach and intestines not working normally, and movement of food through the stomach slows or stops. Patients with gastroparesis typically exhibit symptoms of nausea and/or vomiting and gastric discomfort. They may complain of bloating or a premature or extended feeling of fullness (satiety). The symptoms of gastroparesis are the result of reduced gastric motility. Gastroparesis generally results in patients reducing food intake and subsequently losing weight.

Electrical stimulation of the gastrointestinal tract has been proposed as a mechanism for treating morbid obesity. Table 1 below lists examples of documents that disclose techniques for electrical stimulation of the gastrointestinal tract for the treatment of obesity. These disclosures suggest that disruption in the normal stomach motility, which may then cause symptoms of gastroparesis, may be useful in the treatment of obesity.

TABLE 1

| Pat. No. | Inventors | Title |
|---|---|---|
| 20020072780 | Foley | Method and apparatus for intentional |
| | | impairment of gastric motility and /or |
| | | efficiency by triggered electrical stimu- |
| | | lation of the gastrointestinal tract with |
| | | respect to the intrinsic gastric electrical |
| | | activity |
| 5,836,994 | Bourgeois | Method and apparatus for electrical |
| | | stimulation of the gastrointestinal tract |
| 5,995,872 | Bourgeois | Method and apparatus for electrical |
| | | stimulation of the gastrointestinal tract |
| 6,091,992 | Bourgeois | Method and apparatus for electrical |
| | | stimulation of the gastrointestinal tract |
| 6,104,955 | Bourgeois | Method and apparatus for electrical |
| | | stimulation of the gastrointestinal tract |
| 6,115,635 | Bourgeois | Method and apparatus for electrical |
| | | stimulation of the gastrointestinal tract |
| 6,216,039 | Bourgeois | Method and apparatus for treating |
| | | irregular gastric rhythms |
| 6,327,503 | Familoni | Method and apparatus for sensing and |
| | | stimulating gastrointestinal tract |
| | | on-demand |
| 5,423,872 | Cigiana | Process and Device for Treating Obesity |
| | | and Syndromes Relates to Motor |
| | | Disorders of the Stomach of a Patient |
| 6,542,776 | Gordon et al. | Gastric Stimulator and Method for |
| | | Installing |
| 6,606,523 | Jenkins | Gastric Stimulator Apparatus and Method |
| | | for Installing |
| 6,615,084 | Cigiana | Process for Electrostimulation Treatment |
| | | of Morbid Obesity |

All documents listed in Table 1 above are hereby incorporated by reference herein in their respective entireties. As those of ordinary skill in the art will appreciate readily upon reading the Summary of the Invention, Detailed Description of the Preferred Embodiments and Claims set forth below, many of the devices and methods disclosed in the patents of Table 1 may be modified advantageously by using the techniques of the present invention.

### SUMMARY

In general, the invention is directed to medical devices and methods for electrical stimulation of the stomach of a patient by monitoring one or more physiological parameters that indicate the activity of the stomach, and applying electrical stimulation to the stomach to induce symptoms of gastroparesis in response to the monitored parameters. The induced symptoms of gastroparesis may reduce a patient's desire to consume large portions of food and thus provide an effective treatment for obesity.

The symptoms of gastroparesis suggest that some effects of inducing gastroparetic symptoms, rather than gastroparesis itself, may be beneficial as a therapy for obesity, if the symptoms are properly modulated. More significantly, the symptomology of gastroparesis, if associated with gastric activity, may provide an effective form of biofeedback therapy for the treatment of obesity, discouraging a patient form consuming excessive quantities of food.

Various embodiments of the present invention provide solutions to one or more problems existing in the prior art with respect to prior techniques for treatment of obesity. These problems include the lack of feedback to the patient about his stomach activity. Natural feedback mechanisms, such as the normal sensation of fullness following a meal, may be insufficient for a patient to regulate his own behavior. In addition, natural feedback mechanisms may be inadequate to control a patient's behavior. An obese patient, for example, may continue to consume food after being full because of a delay between onset of fullness and the onset of the sensation of fullness.

As a further problem, a diabetic patient as well as non-diabetic patients may be unable to readily comprehend the size or composition of a meal, which over time may contribute to weight gain and eventually to an obese condition. Blood glucose fluctuates in both diabetic and non-diabetic patients in response to ingested food. See Tanenberg R J, Pfeifer MA Continuous glucose monitoring system: a new approach to the diagnosis of diabetic gastroparesis, Diabetes Technol. Ther. 2000, 2 Suppl. 1:S73-80. The amount a blood glucose fluctuation from baseline can be used to assess the caloric content of an ingested meal and may be used by the patient as feedback to adjust or control food intake.

Additional problems arise when electrical stimulation is applied to the stomach. In particular, the inability to provide feedback of stomach activity undermines efforts to automatically control delivery of electrical stimulation to the stomach. In particular, without an indication of stomach activity such as food intake, it is difficult to determine a precise time for delivery of electrical stimulation to induce symptoms of gastroparesis.

Consequently, using electrical stimulation of the gastrointestinal tract to induce gastroparesis has significant drawbacks. The treatment typically is applied to patients all of the time, which may result in adverse health effects associated with continuous disruption in normal stomach motility. However, manual techniques of inducing gastroparesis only during times of expected gastric activity associated with eating food require patients to manually induce undesirable symptoms for the treatment of their obesity.

Various embodiments of the present invention are capable of solving at least one of the foregoing problems. For example, the invention may provide features for treatment of obesity by providing gastric stimulation in response to sensed gastric activity, permitting more controlled delivery of gastric stimulation in an automated manner. Distension of the stomach is one example of a physiological parameter indicating activity of the stomach, such as food intake, and can be monitored and then used to trigger delivery of gastric stimulation only when gastric activity is sensed.

In this manner, a device and method in accordance with the invention is capable of providing biofeedback to the patient by inducing symptoms of gastroparesis using electrical stimulation of the gastric tract in response to sensed gastric activity. The symptoms of gastroparesis may discourage patients from consuming large quantities of food. As a result, inducing symptoms in response to detection of gastric activity may permit more targeted delivery of electrical stimulation at appropriate times incident to food intake, and result in a reduction of the amount of food consumed by a patient.

When embodied as an implantable device, the invention includes features including one or more sensors to sense a physiological parameter indicative of gastric activity such as food intake. The invention also includes a processor that generates gastric electrical stimulation to the patient as a function of the sensed physiological parameter.

The processor monitors one or more physiological parameters and may measure various characteristics of a physiological parameter, such as a rate of change, amplitude, duration, intensity and concentration. The processor can evaluate whether a characteristic should be brought to the attention of the patient, e.g., for manual actuation of techniques for inducing symptoms of gastroparesis, or automatically direct application of gastric electrical stimulation as a function of the measured characteristic. The processor may respond to extreme distension of the stomach of a particular patient, for example, by directing application of gastric electrical stimulation, but withhold application of electrical stimulation when mild distension is sensed.

In comparison to known implementations of gastric stimulation used for the treatment of obesity, various embodiments of the invention may provide one or more advantages. The invention provides gastric electrical stimulation in response to sensed gastric activity rather than continuous gastric electrical stimulation or patient activated gastric electrical stimulation. The ability to automatically control delivery of electrical stimulation in response to sensed gastric activity eliminates the need to delivery electrical stimulation continuously, alleviating potentially adverse health effects associated with continuous disruption in normal stomach motility. As such, the invention may provide more effective treatment for obesity while providing considerable freedom and enjoyment of life for the patient. In various embodiments, the patient can use the invention to obtain biofeedback responsive to consumption of food and to provide an effective mechanism to exercise control over his or her own health and well-being.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIGS. 1A and 1B are diagrams illustrating devices for monitoring activity of the stomach and providing electrical stimulation to patient responsive to stomach activity.

FIG. 2 is a block diagram illustrating constituent components of an embodiment of a device as depicted in FIG. 1.

FIG. 3 is a diagram illustrating an exemplary electrical stimulation signal applied to a patient's gastrointestinal tract to induce symptoms of gastroparesis.

FIG. 4 illustrates a graphical representation of an exemplary sensed physiological parameter over a period of time.

FIG. 5 illustrates a graphical representation of another exemplary sensed physiological parameter over a period of time.

FIG. 6 is a flow diagram illustrating a technique for generating a communication or controlling delivery of electrical stimulation as a function of a sensed physiological parameter.

FIG. 7 is a flow diagram illustrating a further technique for controlling delivery of electrical stimulation as a function of a sensed physiological parameter.

### DETAILED DESCRIPTION

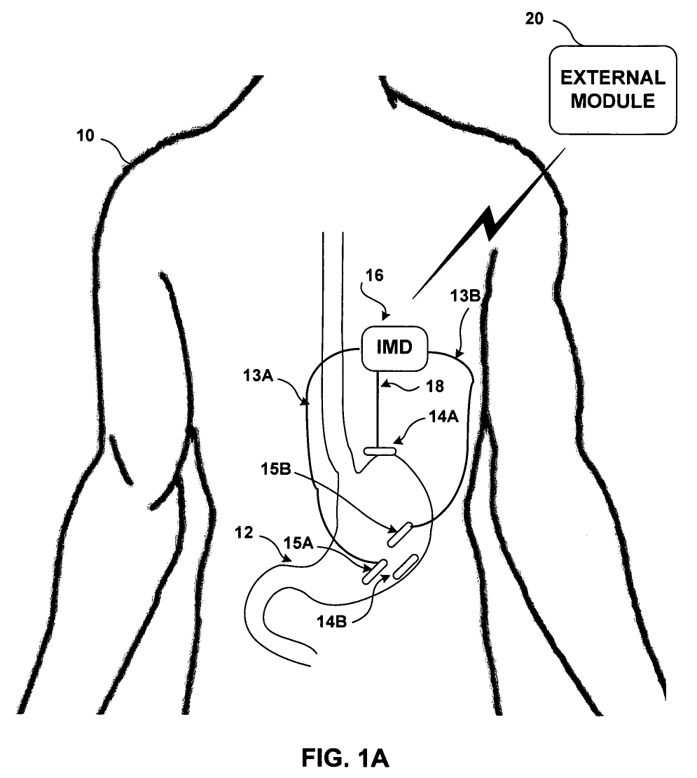

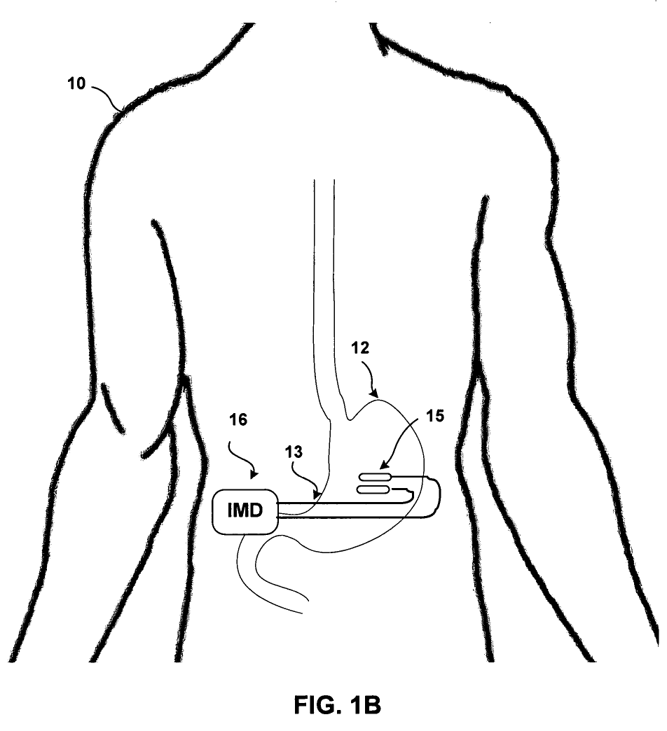

FIG. 1A is a block diagram illustrating a view of a torso of a patient 10, in which stomach 12 is visible. FIG. 1A further illustrates devices for monitoring one or more physiological parameters that indicate the activity of stomach 12, and applying electrical stimulation to the stomach to induce symptoms of gastroparesis in response to the monitored parameters.

Physiological parameters such as blood glucose or insulin concentration, core body temperature, distension of the stomach, pH level of the stomach and various plasma enzymes may provide an indication of stomach activity within patient 10. In particular, each of these parameters varies as a function of food intake. As a result, one or more of these physiological parameters can be monitored to detect food intake, and thereby trigger a response, such as delivery of electrical stimulation to stomach 12 of patient 12 to induce symptoms of gastroparesis, and thereby influence further food intake by the patient.

In the example of FIG. 1, sensors 14A and 14B (hereinafter referred to as “sensors 14”) sense physiological activity of stomach 12. Sensor 14A is implanted in the body of patient 10, but is external to stomach 12. Sensor 14A is coupled to an implantable medical device (IMD) 16 by a lead 18. Sensor 14B, by contrast, is deployed inside stomach 12, and may communicate with IMD 16 wirelessly. The invention is not limited to deployment of two sensors, nor is the invention limited to deployment of sensors at the sites shown in FIG. 1A.

Sensor 14 may be any sensor that senses or responds to any physiological parameter that reflects activity of stomach 12, such as activity incident to food intake, i.e., meal ingestion. In some embodiments, sensor 14 includes one or more electrodes to detect gastric electrical activity, trans-abdominal impedance, or other electrical indicators of stomach activity. In other embodiments, sensor 14 includes a chemical sensor that detects blood glucose, stomach acid, or other chemical indicators of stomach activity. In further embodiments, sensor 14 includes one or more mechanical sensors to detect motion of stomach 12, distension of stomach 12, or other mechanical indicators of stomach activity. The invention is not limited to mechanical, chemical and electrical sensors, however, but includes other types of sensor as well, such as temperature sensors or acoustic sensors. Additional details regarding automatically obtaining notification of gastric activity is described in commonly assigned U.S. patent application to Starkebaum, entitled “GASTRIC ACTIVITY NOTIFICATION,” Ser. No. 10/698,115, filed Nov. 1, 2003 (Attorney Docket No. P-9903.00) which is hereby incorporated by reference herein.

Physiological parameters sensed by sensor 14 are supplied to IMD 16. IMD 16 measures a characteristic of a physiological parameter sensed by sensor 14. For a sensed physiological parameter, IMD 16 may track the parameter over time, measuring the rate of change of the parameter, for example, the amplitude of the parameter, the duration of the parameter, the intensity or concentration of the parameter, or other qualities. In response, IMD 16 may control application of electrical stimulation to the gastric tract, including stomach 12. Simulation electrodes 15A, 15B (hereinafter referred to as “stimulation electrodes 15”) are connected to IMD 16 using electrical leads 13A and 13B (hereinafter referred to as “leads 13”). Stimulation electrodes 15A, 15B may be affixed to an external surface of the stomach via sutures, surgical adhesives, or the like. Experimental results have shown that stimulation electrodes 15 may be implanted at many locations within the stomach as it is believed that the electrical stimulation couples to the vagal nerve to transmit signals to a patient's brain. As such, any location in which the electrical coupling to the nerve is possible may be used.

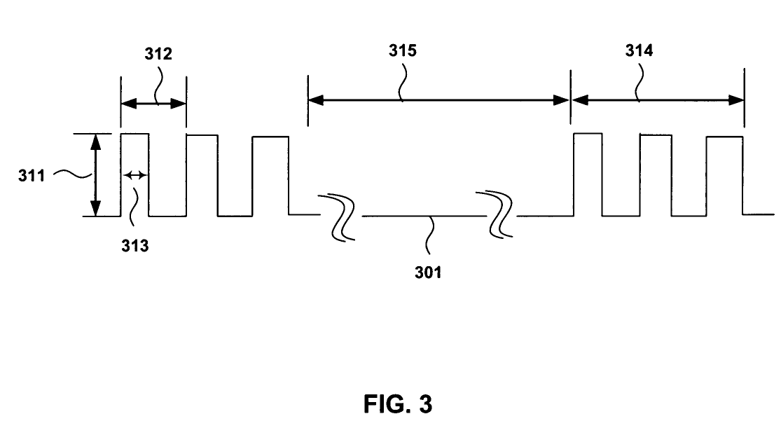

IMD 16 provides electrical stimulation of the stomach 12 through stimulation electrodes 15 to induce symptoms of gastroparesis, such as nausea and gastric discomfort, as part of treatment for obesity. Based upon experimental work associated with gastric stimulation for gastroparesis, these symptoms associated with gastroparesis may be induced using a stimulation signal described in more detail with respect to FIG. 3.

IMD 16 may provide electrical stimulation to stimulation electrodes 15 to induce the desired symptoms during a time period in which IMD 16 detects gastric activity using sensors 14. As such, obesity patients experience uncomfortable symptoms during time periods associated with eating and may alter their behavior to eat less food. In addition, IMD 16 may alter the length of time during which electrical stimulation is provided to discourage consumption of larger portions of food. For example, IMD 16 may detect an excessively large portion of food being present in the stomach 12 by examining an amplitude of parameters obtained from sensors 14. In addition, IMD 16 may detect an excessively large portion of food by measuring an amount of time required for the stomach 12 to process the portion of food into lower portions of the gastrointestinal tract. When IMD 16 detects existence of such conditions, IMD 16 may provide electrical stimulation for a longer period of time than normal. Using this approach, IMD 16 may provide negative biofeedback associated with consumption of larger portions of food.

The electrical stimulation provided by IMD 16 may be activated for a variety of ways once gastric activity has been detected. In one embodiment, electrical stimulation may be initiated by IMD 16 immediately upon detection of gastric activity as symptoms are induced quickly after initiation of electrical stimulation. Once initiated, the electrical stimulation may continue for a fixed period of time, or may continue until IMD 16 no longer detects gastric activity. In other embodiments, the electrical stimulation may be initiated at various times of the day associated with meals. In yet other embodiments, the electrical stimulation may begin upon detection of gastric activity and end at a point in time following the detection of the end of gastric activity, where the point in time is determined by an estimate of a particular patient's need for appetite suppression. Any combination of these techniques or any other similar techniques may be used with out departing from the spirit and scope of the present invention.

Similarly, IMD 16 may detect the number of times electrical stimulation is provided within a 24 hour period of time regardless of the size of portions detected. When the number of times electrical stimulation is provided exceeds a predetermined number, additional and extended periods of electrical stimulation may be provided by IMD 16 to induce undesirable symptoms in an attempt to discourage a patient from eating more than a predetermined number of times each day. The undesirable symptoms serve as negative biofeedback, discouraging the patient from consuming additional food. The length of an extended period of electrical stimulation may increase with each additional detection of gastric activity to increase an amount of negative biofeedback provided to a patient that is associated with an undesired consumption of food.

IMD 16 may also generate a communication to patient 10 as a function of the measurement. External module 20 may be a device dedicated to presenting information pertaining to stomach activity, or external device 20 may be a general purpose device such as a pager, cellular telephone, or personal digital assistant (PDA). As shown in FIG. 1, IMD 16 communicates wirelessly with external module 20 via RF telemetry, but the communication may also be transmitted via a transcutaneous wired or optical connection.

When sensor 14B comprises a mechanical sensor that senses distension of stomach 12, MD 16 measures and records the sensed distension and generates a communication to the patient based on the measurement. The communication, which is transmitted to external module 20, may include information concerning the timing of the distension, the rate of distension, the magnitude of the distension, and the like. Of course, the communication to the patient 20 may be used with any sensor 14 generating information useful to patient 20 in providing care for one's well being.

IMD 16 may consist of a pair of stimulation electrodes 15. The stimulation electrodes 15 may consist of intramuscular electrodes or surface electrodes. Intramuscular electrodes are placed in the muscle wall of the stomach, preferably in the circular muscle layer. These stimulation electrodes may be inserted either from the serosal aspect of the stomach (i.e., the from the outer surface) or from the musosal aspect (i.e., from the inside side of the stomach. Surface electrodes may be attached to the serosa or mucosa, though the serosa is preferred.

Stimulation electrodes 15, such as the model 4351 stimulation electrodes and leads manufactured by Medtronic, Inc., and are connected to IMD 16. IMD 16 may be an implanted stimulator, such as model 7425 or model 3116 implantable stimulator manufactured by Medtronic, Inc.

The pair of stimulation electrodes 15 may be placed in the muscle wall of the stomach using standard surgical practices including laparotomy or laparoscopy, as shown in FIG. IB. The pair of stimulation electrodes 15 may be positioned anywhere in the stomach, but typically are placed along either the greater curvature or lessor curvature. IMD 16 may be positioned subcutaneously in the abdominal wall, typically in the right mid quadrant and may then be programmed by radio-telemetry link to the appropriate stimulation parameters using an external module 20. FIG 1B illustrates a pair of stimulation electrodes 15 positioned along greater curvature. In other embodiments, stimulation electrodes capable of being positioned either on the stomach wall or embedded within the muscle wall may be used without departing from the spirit and scope of the present invention. Attachment of the stimulation electrodes 15 may be accomplished by means of sutures, surgical clips, or screws, such as are typically used with screw-in leads.

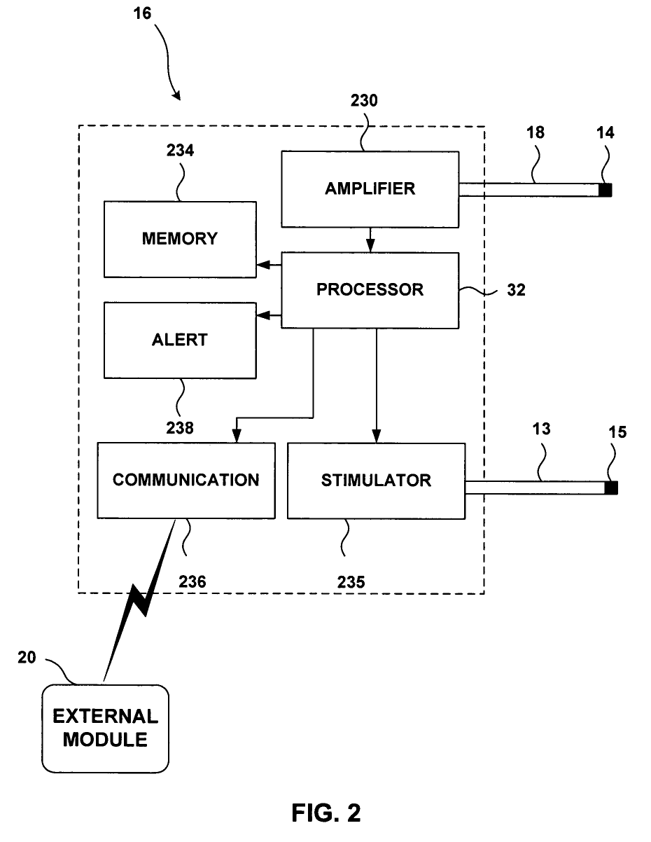

FIG. 2 is a block diagram illustrating device 16 in greater detail in accordance with an embodiment of the invention. In FIG. 2, IMD 16 is coupled to a sensor 14 by a lead 18. An amplifier 230 receives signals detected by sensor 14. The signals detected by sensor 14 are representative of physiological parameters relating to gastric activity, such as food intake. Amplifier 230 amplifies and filters the received signals and supplies the signals to a processor 232. Processor 232 processes the received signals, and analyzes a physiological parameter of interest.

The received signal is typically converted to digital values prior to processing by processor 232, and stored in memory 234. Memory 234 may include any form or volatile memory, non-volatile memory, or both. In addition to data sensed via sensor 14, memory 234 may store records concerning measurements of detected physiological parameters, communications to patient 10 or other information pertaining to operation of MD 16. Memory 234 may also store information about patient 10, and thresholds for comparison to the physiological parameters obtained by sensor 14. In addition, processor 232 is typically programmable, and programmed instructions reside in memory 234.

Processor 232 determines whether to direct application of electrical stimulation to patient 10 based upon the measurements indicated by sensor 14. As shown below, processor 232 may compare a parameter, or one or more characteristics of a parameter, to a threshold, and control a stimulator 235 to apply an electrical stimulation signal via stimulation electrode 15. Stimulator 235 includes suitable pulse generation circuitry for generating a voltage or current waveform with a selected amplitude, pulse width, and frequency sufficient to induce symptoms of gastroparesis. In response to a control signal from processor 232, the electrical stimulation signal generated by stimulator 235 is applied to a patient's gastrointestinal tract when the threshold is surpassed. This electrical stimulation signal may be generated until processor 232 detects a cessation of gastric activity using the physiological parameter of interest detected by sensor 14, at which time processor 232 controls stimulator 235 to stop delivery of the electrical stimulation. Processor 232 may also record the occurrence of electrical stimulation within memory 234 for use in determining whether additional electrical stimulation is desired to increase an amount of negative biofeedback provided to the patient 10.

For example, processor 232 stores an occurrence of electrical stimulation in memory 234. The next time processor 232 determines electrical stimulation is needed, processor 232 may search memory 234 to determine when the prior electrical stimulation occurred in order to estimate whether electrical stimulation for an extended period of time may be useful. If a patient 20 consumes food on more occasions than may be specified in a particular treatment plan for obesity, electrical stimulation for extended periods of time beyond a baseline time period may be useful in encourage patients to reduce the number of occasions in which food is consumed. Similarly, a record of the prior occurrence of electrical stimulation may be used to ensure that a minimum amount of time passes between the detection of gastric activity. When gastric activity is detected before the minimum amount of time has passed, electrical stimulation may also be provided for an extended period of time to encourage patient 20 from eating food as often.

When processor 232 controls stimulator 235 to deliver the electrical stimulation signal, processor 232 may also convey the communication to patient 10 in a number of ways. IMD 16 may include, for example, a communication module 236 to wirelessly transmit the communication to external module 20. In this manner, patient 10 may be notified that IMD 16 has detected intake of food, and may apply electrical stimulation to stomach 12 to induce symptoms of gastroparesis shortly. The notification may be generated by external module 30 in the form of a visible or audible notification, e.g., emitted by a light, LED, display, or audio speaker. A visible notification may be presented as text, graphics, one or more blinking lights, illumination of one or more lights, or the like. An audible notification may take the form of an audible beep, ring, speech message, or the like. In addition to transmitting a communication to an external module 20, communication module 236 may be configured to wirelessly transmit information about the history or status of IMD 16 to the physician for patient 10.

In addition, or in the alternative, IMD 16 may include an alert module 238 that is implanted in the body of patient 10. When activated by processor 232, alert module 238 can notify patient 10 directly without an external module. Alert module 238 may, for example, notify patient 10 audibly or by vibration. For example, alert module 238 may take the form of a piezoelectric transducer that is energized in response to a signal from processor 232 in order to emit a sound or vibration. In each case, patient 10 receives a communication that IMD 16 has detected a physiological parameter indicative of intake of food, and that symptoms of gastroparesis may be imminent.

FIG. 3 is a diagram illustrating an exemplary electrical stimulation signal 301 applied to a patient's gastrointestinal tract to induce symptoms of gastroparesis. Based upon experimental work associated with gastric stimulation of gastroparesis, an electrical stimulation signal 301 is believed to induce symptoms of gastroparesis by activating an afferent pathway to patient 10 brain the via the patient's vagal nerve. This electrical stimulation using electrical stimulation signal of FIG. 3 typically does not cause disruption in the normal stomach motility.

Electrical stimulation signal 301 possesses a set of signal parameters including amplitude 311, signal frequency 312, pulse width 313, and a duty cycle with an on period 314 and an off period. Experimentally, preferred values for this set of signal parameters are amplitude 311=approximately 0.1 to 10 mA, and preferably approximately 5 mA, signal frequency 312=approximately 10 to 250 Hz, and preferably approximately 14 Hz, pulse width=approximately 100 to 100 microseconds, and preferably approximately 330 microseconds, and a duty cycle with an on period 314=approximately 0.1 to 0.5 seconds, and preferably approximately 0.1 seconds and an off period=approximately 1 to 10 seconds, and preferably approximately 5 seconds. Additional details regarding characteristics of an electrical stimulation signal useful for inducing symptoms of gastroparesis is described in commonly assigned U.S. patent application to Starkebaum, entitled “GASTRIC STIMULATION FOR ALTERED PERCEPTION TO TREAT OBESITY,” Ser. No: ______, filed Jan. 30, 2004 (Attorney Docket No. P-9902.00) which is hereby incorporated by reference herein in its entirety.

In accordance with the invention, an electrical stimulation waveform as described with reference to FIG. 3 is applied following detection of a sensed physiological parameter that exceeds a particular threshold, and thereby indicates recent, current or imminent ingestion of food by patient 10. Again, one or more characteristics of a physiological parameter, such as a rate of change, amplitude, duration, intensity or concentration may be compared to an applicable threshold to detect food intake. In some embodiments, multiple thresholds for a single parameter, or multiple thresholds for different parameters may be evaluated to provide a correlation that provide an indication of food intake with greater certainty.

In various embodiments, the electrical stimulation signal may be applied immediately following detection of food intake, or after a predetermined period of time following detection of food intake, and may be applied for different periods of time. The period of time at which, and for which, the electrical stimulation is applied may be fixed, or vary according to the level of a physiological parameter characteristic of interest. If a parameter such as distension indicates a large meal has already been ingested, electrical stimulation may be applied immediately or in a shorter period of time following detection of food intake, and may be applied for a longer period of time. Also, in some embodiments, electrical stimulation parameters may be adjusted to bring about a response more quickly, e.g., induce symptoms of gastroparesis more quickly, if a larger meal is detected.

Similar adjustments in time and stimulation parameters may be applied if the physiological parameter indicates continued ingestion of food despite application of the electrical stimulation signal. In other words, continued food intake may be countered by more potent electrical stimulation in some cases. In each case, electrical stimulation can be applied at a particular time relating to gastric activity of the patient 10, and for a limited period of time. Consequently, there is no need to deliver electrical stimulation continuously, and undesirably subjecting the patient to continued symptoms of gastroparesis. Hence, patient 10 may enjoy a better quality of life as a result of targeted delivery of electrical stimulation.

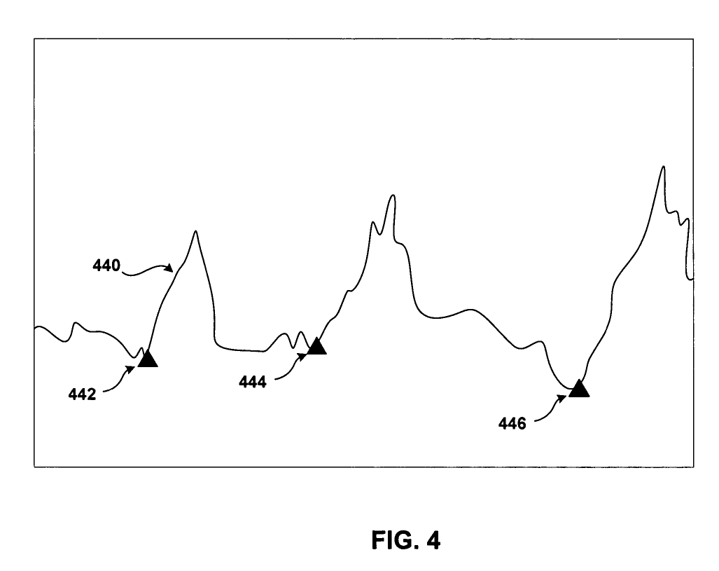

FIG. 4 illustrates analysis of an exemplary physiological parameter. FIG. 4 includes a graphical representation 440 of the blood glucose for patient 10 sensed by sensor 14 over a period of time. Monitoring blood glucose is important for a patient 10 who has been diagnosed with diabetes, and who treats his condition by regulating his diet and by administering insulin injections. FIG. 4 is demonstrative and conceptual, and does not represent actual measured data. Sensor 14 may sense blood glucose levels chemically, optically, with infrared light, or using any other sensing technique.

Initially, the blood glucose level is stable and at a baseline level. Blood glucose level generally changes with stomach activity, however. In particular, ingestion of a meal typically causes blood glucose levels to rise. After consumption of meals, as indicated by reference numerals 442, 444 and 446, sensor 14 senses a substantial increase in blood glucose. Processor 232 of IMD 16 measures a characteristic of the physiological parameter, such as the amplitude, rate of change, duration of elevated glucose level, or any other characteristic. Further, processor 232 compares the measured characteristic to a threshold value stored in memory 234 and controls generation of electrical stimulation signal 301 by stimulator 235 when the measured characteristic surpasses the threshold. In this manner, IMD 16 induces symptoms of gastroparesis discourages patient 10 from ingesting more food. Processor 232 may also generate a communication to external module 20 to notify patient 10 of his current condition. The communication can further notify patient 10 as to what action patient 10 ought to take to treat his current condition, such as insulin injections.

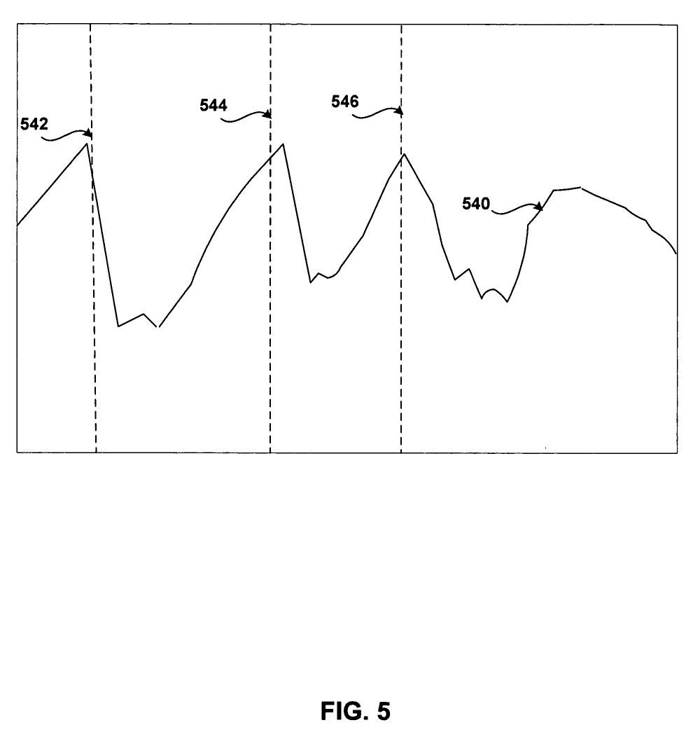

FIG. 5 illustrates analysis of another exemplary physiological parameter. FIG. 5 includes a graphical representation 540 of the plasma levels for ghrelin, a blood enzyme, for patient 10 over a period of time. Ghrelin is a hormone secreted by glands containing parietal cells located principally in the mucosal lining of the stomach. Recent studies suggest that ghrelin is a potent appetite stimulant in animals and man when administered orally. Plasma ghrelin levels have been shown to fluctuate over a 24 hour cycle. In particular, plasma ghrelin levels are elevated before meals, and fall dramatically after meals. FIG. 5 is demonstrative and does not represent actual measured data. As one example, ghrelin levels may be sensed using a blood test with results entered into external module 20 for transmission to IMD 16.

Initially, the ghrelin level is stable and at a baseline level. Ghrelin level generally changes with stomach activity, however. In particular, ingestion of a meal typically causes ghrelin levels to fall. Experimental results have also shown that ghrelin levels typically peak at time periods immediately prior to normal consumption of meals, as indicated by reference numerals 542, 544 and 546. As such, detection of ghrelin levels may be used as a predictor of consumption of food prior to actual consumption.

Processor 232 of IMD 16 may use this data to begin electrical stimulation to induce gastroparesis symptoms to discourage and reduce consumption of food. Processor 232 may analyze a characteristic of the physiological parameter, such as the amplitude, rate of change, duration of elevated ghrelin level, or any other characteristic. Further, processor 232 compares the measured characteristic to a threshold value stored in memory 234 and generates electrical stimulation signal 301 when the measured characteristic surpasses the threshold. Advantageously, detection of a parameter such as ghrelin level may provide an advance indication of food intake, and permit processor 232 to control delivery of electrical stimulation prior to ingestion of a meal. Hence, this parameter may permit preemptive action to induce symptoms of gastroparesis prior to a meal, and thereby limit intake by patient 10.

The criteria for generating electrical stimulation signal 301 may vary from patient to patient. For some patients, a sharp increase in a measured single physiological parameter may result in the generation of electrical stimulation signal 301. In other patients, a sharp increase is of less concern than a high amplitude or peak value of the physiological parameter. In a further set of patients, the duration of elevation for a measured physiological parameter may be of special concern. The invention provides for measuring a variety of characteristics of a single physiological parameter. Additional details regarding automatically obtaining notification of gastric activity is described in commonly assigned U.S. patent application to Starkebaum, entitled “GASTRIC ACTIVITY NOTIFICATION,” Ser. No. 10/698,115, filed Nov. 1, 2003 (Attorney Docket No. P-9903.00).

In addition, processor 232 may measure a characteristic of one physiological parameter as a function of another physiological parameter. There is a relationship, for example, between the blood glucose levels following a meal and the caloric content of the meal. By analysis of blood glucose levels, processor 232 can estimate the caloric intake of patient 10. In an obese patient, an estimate of caloric intake may be of greater interest than blood glucose concentration. For example, the estimate for caloric intake may be useful in determining a length of time electrical stimulation is provided to a patient. When processor 232 determines a meal having an estimated caloric intake greater than a predetermined threshold, electrical stimulation may be provided for an extended period of time as compared to the amount of electrical stimulation provided when the estimated caloric intake is less than the predetermined threshold.

In the event the measured characteristic surpasses the applicable threshold, processor 232 controls application of electrical stimulation signal 301 and a communication via communication module 236 and external module 20, or alert module 238, to notify patient 10. Patient 10 may respond by, for example, self-administering medication, ceasing eating, or seeking medical attention. IMD 16 continues to monitor the physiological parameter to determine whether the condition is being addressed.

Similar techniques may be applied to physiological parameters other than blood glucose and ghrelin to reflect stomach activity. Accordingly, the invention provides a convenient vehicle for the monitoring and treatment of obesity, diabetes, eating disorders, and the like. In addition, the invention allows the patient to obtain information about his condition and to exercise control over his own health and well-being. In addition, the embodiments of the present invention disclosed herein utilize electrical stimulation applied to a patient's gastrointestinal tract to induce symptoms of gastroparesis as a method of providing biofeedback as a result of sensed gastric activity. Of course, one skilled in the art will appreciate that other forms of stimulation may also be useful in providing biofeedback without departing from the spirit and scope of the present invention.

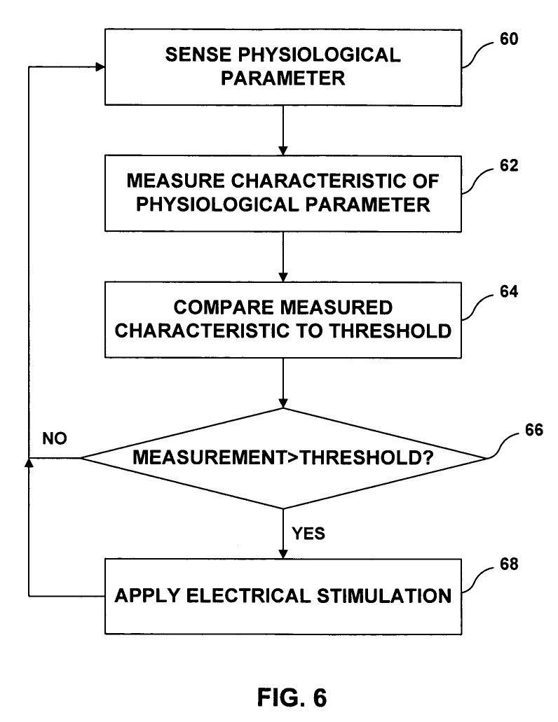

FIG. 6 is a flow diagram illustrating a technique for monitoring one or more physiological parameters that reflect stomach activity. Processor 232 receives data concerning a physiological parameter that reflects stomach activity from sensor 14 (60). Sensor 14 may respond to any of several electrical, mechanical, chemical or other physiological parameters.

Processor 232 processes the data received from sensor 14 and measures one or more characteristics as a function of the sensed physiological parameter (62). The measured characteristic can be a characteristic of the physiological parameter itself, such as the concentration of blood glucose or the magnitude of stomach distension. The measured characteristic can also be a characteristic of a related physiological parameter, such as a measurement of caloric intake as a function of blood glucose levels.

Processor 232 compares the measured characteristic to a threshold value (64) stored in memory 234. When the measured characteristic surpasses the threshold, processor 232 controls application of a electrical stimulation signal in order to induce gastroparesis symptoms providing biofeedback to patient 10 (68). When the measured characteristic does not surpass the threshold, processor 232 may continue to monitor the physiological parameters. In some implementations, a measurement will “surpass” a threshold when the measurement is above the threshold, and in other implementations, the measurement will “surpass” a threshold when the measurement is below the threshold.

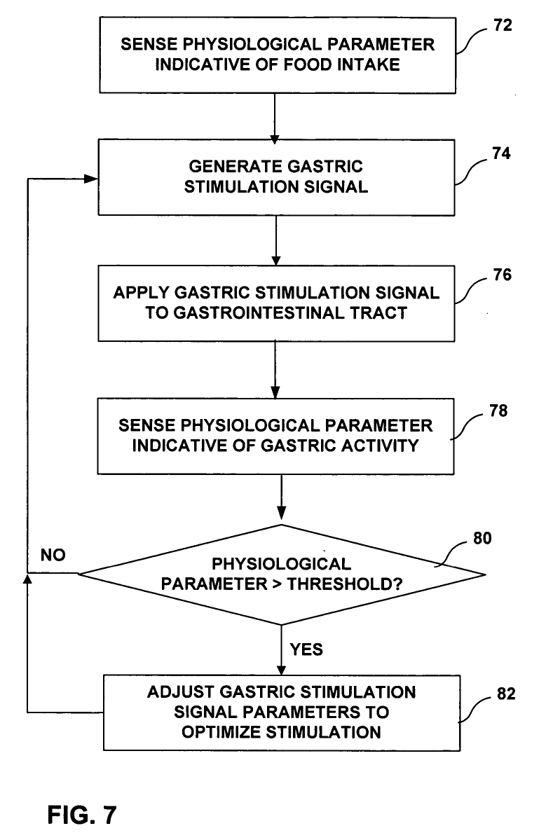

FIG. 7 is a flow diagram illustrating another technique for monitoring one or more physiological parameters that reflect stomach activity, and controlling electrical stimulation in response to the indicated stomach activity. Processor 232 receives data concerning a physiological parameter that reflects stomach activity such as food intake from sensor 14 (72). Sensor 14 may respond to any of several electrical, mechanical, chemical or other physiological parameters.

Processor 232 processes the data received from sensor 14 and measures one or more characteristics as a function of the sensed physiological parameter. Processor 232 then controls the generation of an electric stimulation signal, using stimulator 235, using the sensed physiological parameter in order to induce gastroparesis symptoms to patient 10 (74). The measured characteristic can be a characteristic of the physiological parameter itself, such as the concentration of blood glucose or the magnitude of stomach distension. The measured characteristic can also be a characteristic of a related physiological parameter, such as a measurement of caloric intake as a function of blood glucose levels. In some embodiments, the electric stimulation signal has electrical signal parameters selected in order to induce desired symptoms without reducing normal stomach motility.

Processor 232 controls transmission of the electric stimulation signal from stimulator 235 to patient 10. In particular, processor 232 controls stimulator 235 to apply the electrical stimulation signal (76) to the gastrointestinal tract of patient 10. Again, the stimulation parameters may be selected to induce the desired symptoms without substantially reducing normal stomach motility. Techniques for inducing symptoms of gastroparesis without substantially reducing normal stomach motility are described, for example, in the above-referenced patent application to Starkebaum, filed concurrently herewith. Processor 232 also may control stimulator 235 to initiate and terminate transmission of the electric stimulation signal in response to external commands received from external module 20 as discussed above.

As further shown in FIG. 8, upon generation of a gastric electrical stimulation signal (74), and application of the signal to the gastrointestinal tract (76), processor 232 senses another physiological parameter indicative of gastric activity (78) to determine whether the stimulation signal is producing a desired effective in inducing symptoms of gastroparesis, whether the objective is to actually induce gastroparesis and thereby disrupt stomach motility, or merely to induce symptoms of gastroparesis without substantially disrupting stomach motility. In either case, processor 232 compares the physiological parameter to an applicable threshold (80), and adjusts the gastric stimulation parameters, if necessary, to achieve optimal stimulation (82).

The physiological parameter may be obtained via sense electrodes or other types of transducers capable of providing an indication of gastric activity, e.g., on a continuous or periodic basis during delivery of stimulation. The physiological parameter may be any of a variety of parameters such as an electrical signal level, frequency, duty cycle, or the like, which is indicative of stomach motility. Alternatively, the morphology of a physiological waveform may be analyzed and compared to reference points or a signal waveform template to indicate stomach motility. In either case, if gastric activity does not compare favorably to a predetermined threshold or other criteria, modifications to the stimulation signal may include increasing or reducing signal amplitude, signal pulse width, signal frequency, and signal duty cycle, so that desired results are achieved. Alternatively, modifications may include termination of transmission of the electrical stimulation signal completely if undesired gastric activity is detected from the sensed physiological parameter.

Hence, in accordance with the embodiment of FIG. 7, IMD 16 also may operate in a closed loop mode, not only in response to food intake, but also in response to feedback indicative of the effectiveness of the electrical stimulation in achieving desired symptoms of gastroparesis. In particular, if sensed physiological parameters indicate that symptoms of gastroparesis have not yet been achieved, the electrical stimulation parameters may be adjusted to provide more intense stimulation. Alternatively, if sensed physiological parameters indicate earlier onset of symptoms, the duration or intensity of the electrical stimulation may be reduced. As a further alternative, if the physiological parameter indicates that stimulation has undesirably affected stomach motility, the electrical stimulation can be adjusted or terminated to restore normal motility. Hence, IMD 16 may be responsive to physiological parameters indicative of intake of food to initiate electrical stimulation, as well as physiological parameters indicative of onset and status of symptoms to adjust the electrical stimulation parameters for optimum stimulation.

A variety of physiological parameters may be sensed to obtain an indication of gastric activity, including effectiveness of stimulation in achieving desired symptoms. It is known, for example, that symptoms such as nausea are associated with certain physiological parameters that may be sensed to control the operation of IMD 16. In alternate embodiments of IMD 16, these physiological parameters may be sensed and then used to control generation of the electric stimulation signal.

In one case, it has been shown that tachygastria i.e., an abnormally fast gastric slow wave, is associated with nausea. See Koch et al., page 198, Chapter 13, Electrogastrography, in Gastrointestinal Motility in Health and Disease, ed. Schuster, Crowell, Koch; B C Deckere, 2^{nd} Edition, 2002. Thus, the appearance of tachygastria sensed from electrodes in the stomach may be used to further control IMD 16.

Various prior gastric stimulation systems monitor an electrical signal from the stomach and then use this sensed electrical signal to control a gastric stimulation system for the purpose of treating gastric arrhythmias and functional gastrointestinal disorders. See commonly assigned U.S. Patents to Bourgeois et al described above for examples of these gastric stimulation systems. Sensing techniques similar to those described by Bourgeois may be used to control a gastric stimulator for the purpose of modulating gastrointestinal symptoms for treatment of obesity. A patient's gastrointestinal tract may be electrically stimulated using IME 16. IMD 16 may also sense a gastric slow wave as described in Bourgeois et al. If an abnormal slow wave is not detected, IMD 16 may adjust stimulation parameters to provide more intense stimulation in an effort to achieve symptoms of gastroparesis, e.g., by increasing or decreasing amplitude, frequency, pulse width, duration, or the like.

In some embodiments, if a sensed slow wave is either too slow or too fast, for example, IMD 16 may be responsive to adjust stimulation parameters accordingly. In humans, the normal gastric slow wave frequency is 3 cycles per minute. As such, a gastric slow wave may be considered indicative of gastroparesis symptoms and disruption of stomach motility if it is abnormally slow, e.g., less than 2.5 cycles per minute. Similarly, a slow wave may be considered abnormally fast when the sensed gastric slow wave is greater than 3.5 cycles per minute.

Additionally, it has been shown that contractile activities in the duodenum and small intestine occur during nausea. See Lacy et al, page 1478, Chapter 10, Manometry, in Gastrointestinal Motility in Health and Disease, ed. Schuster, Crowell, Koch; B C Deckere, 2^{nd} Edition, 2002. Therefore, peristaltic contractions from the duodenum, small intestine, or other regions of the gastrointestinal tract may be measured using strain gauges or other means, and such contractions may be used instead of a gastric slow wave to control adjustment of electric stimulation signal parameters by IMD 16. Other similar physiological parameters may be utilized without departing from the spirit and scope of the present invention.

The invention further encompasses one or more computer-readable media comprising instructions that cause a processor, such as processor 232, to carry out the techniques of the invention. A computer-readable medium includes, but is not limited to, any magnetic or optical storage medium, ROM or EEPROM.

The preceding specific embodiments are illustrative of the practice of the invention. It is to be understood, therefore, that other expedients known to those skilled in the art or disclosed herein may be employed without departing from the invention or the scope of the claims. For example, the present invention further includes within its scope methods of making and using systems as described herein. Furthermore, the invention includes embodiments that use techniques to sense physiological parameters in addition to those specifically described herein.

Moreover, the invention includes embodiments in which IMD 16 is not dedicated to sensing stomach activity and providing gastric stimulation, but performs other functions as well. IMD 16 may include or be integrated with, for example, an implantable drug delivery system such as any of a number of SynchroMed pumps manufactured by and commercially available from Medtronic Inc. In such embodiments, IMD 16 may actively administer therapy, such as by dispensing insulin or medication, in addition to generating a communication to patient 10.

The invention further includes embodiments in which processor 232 measures a characteristic as a function of two or more physiological parameters. For example, processor 232 may estimate caloric intake as a function of stomach distension, as sensed by a mechanical sensor, and blood glucose levels, as sensed by a chemical sensor.

In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts a nail and a screw are equivalent structures.

Many embodiments of the invention have been described. Various modifications may be made without departing from the scope of the claims. These and other embodiments are within the scope of the following claims.

1. A method for providing gastric stimulation responsive to sensed stomach activity of a patient, the method comprising:
sensing a physiological parameter of the patient that changes as a function of activity of a stomach of the patient; and
applying an electrical stimulation signal to the patient as a function of the sensed physiological parameter,
wherein the electrical stimulation signal induces symptoms of gastroparesis in the patient.

2. The method according to claim 1, wherein the physiological parameter includes at least one of a blood glucose concentration, an insulin concentration, a plasma ghrelin concentration, a body temperature, a distension of the stomach, a stomach acid concentration, a gastric electrical activity and a transabdominal impedance.

3. The method according to claim 1, further comprising:
measuring a characteristic of the physiological parameter; and
applying the electrical stimulation signal to the patient as a function of the measurement.

4. The method according to claim 3, wherein applying an electrical stimulation signal comprises applying the electrical stimulation signal for a period of time based on a level of a characteristic of the physiological parameter.

5. The method according to claim 4, further comprising applying the electrical stimulation signal for an increased period of time when the level exceeds a predetermined threshold.

6. The method according to claim 3, wherein the characteristic of the physiological parameter comprises at least one of a rate of change of the physiological parameter, an amplitude of the physiological parameter, a duration of the physiological parameter, an intensity of the physiological parameter and a concentration of the physiological parameter.

7. The method according to claim 3, wherein the characteristic of the physiological parameter is a first characteristic of a first physiological parameter, the method further comprising measuring a second characteristic of a second physiological parameter as a function of the first characteristic.

8. The method according to claim 1, wherein the method further comprises:
generating a communication to the patient in response to a characteristic of the sensed physiological parameter.

9. The method according to claim 8, wherein generating a communication comprises transmitting a wireless communication to an external module.

10. The method according to claim 8, wherein generating a communication comprises presenting notification of electrical stimulation to patient via external module.

11. The method of claim 1, wherein generating the communication comprises activating an implanted alert module.

12. The method according to claim 1, wherein the electrical stimulation signal is applied to the patient for a length of time as a function of the sensed physiological parameter.

13. The method according to claim 1, further comprising adjusting the electrical stimulation signal in response to feedback indicating gastric activity.

14. A system for providing gastric stimulation responsive to sensing feedback to induce symptoms in a patient comprising:
a sensor to sense a physiological parameter of a patient that changes as a function of activity of a stomach of the patient; and
a stimulator to generate an electrical stimulation signal to the patient as a function of the sensed physiological parameter.

15. The system according to claim 14, wherein the system further comprises a processor to analyze the physiological parameter over time for use in generating the electrical stimulation signal.

16. The system according to claim 14, further comprising a communication module to wirelessly transmit the communication to an external module.

17. The system according to claim 14, wherein the system further comprises an implanted alert module to notify the patient of the communication.

18. The system according to claim 14, wherein the sensor comprises a chemical sensor.

19. The system of claim 17, wherein the chemical sensor senses at least one of blood glucose concentration, insulin concentration and stomach acid concentration.

20. The system according to claim 14, wherein the sensor comprises a mechanical sensor.

21. The system of claim 20, wherein the mechanical sensor senses at least one of motion of the stomach and distension of the stomach.

22. The system according to claim 14, wherein the sensor comprises an electrical sensor.

23. The system of claim 22, wherein the electrical sensor senses at least one gastric electrical activity and transabdominal impedance.

24. The system according to claim 14, wherein the processor is implantable in the patient.

25. The system according to claim 14, wherein the processor is further configured to measure a characteristic of the physiological parameter and to compare the characteristic to a threshold.

26. The system according to claim 14, wherein the processor adjusts the electrical stimulation signal in response to feedback indicating gastric activity.

27. A system for providing gastric stimulation responsive to sensing feedback to induce symptoms in a patient comprising:
sensing means to sense a physiological parameter of a patient that changes as a function of activity of a stomach of the patient;
processing means to generate a communication to the patient as a function of the sensed physiological parameter; and
stimulating means to induce symptoms in the patient for treatment of obesity responsive to the processing means.

28. The system according to claim 27, wherein the processing means is further configured to measure a characteristic of the physiological parameter.

29. The system of claim 27, wherein the system further comprises a memory means to data associated with the sensed physiological parameter and the measured characteristic.

30. The system according to claim 27, wherein the system further comprises a communications means to notify the patient of a communications generated by the processing means as a function of the sensed physiological parameter.

31. The system according to claim 27, further comprising means for adjusting the electrical stimulation signal in response to feedback indicating gastric activity.

32. A computer-readable medium comprising instructions that cause a processor to provide gastric stimulation responsive to sensing feedback to induce symptoms in a patient, the instructions causing the processor to:
sense a physiological parameter of a patient that changes as a function of activity of a stomach of the patient; and
control application of an electrical stimulation signal transmitted to the patient for inducing gastroparesis symptoms as a function of the sensed physiological parameter.

33. The medium of claim 32, the instructions further causing the processor to:
measure a characteristic of the physiological parameter; and
control generation of an electrical stimulation signal transmitted to the patient for inducing gastroparesis symptoms as a function of the measurement.

34. The medium according to claim 33, the instructions further causing the processor to:
generate a communication to the patient in response to a characteristic of the sensed physiological parameter.

35. The medium according to claim 34, wherein the processor generates a communication by transmitting a wireless communication to an external module.

36. The medium according to claim 34, wherein the processor generates a communication by presenting notification of electrical stimulation to patient via external module.

37. The medium of claim 33, wherein the processor generates a communication by activating an implanted alert module.

38. The medium according to claim 33, wherein the electrical stimulation signal is applied to the patient for a length of time as a function of the sensed physiological parameter.

39. The medium according to claim 33, wherein the instructions cause the processor to adjust the electrical stimulation signal in response to feedback indicating gastric activity.
